(19) United States
(12) Patent Application Publication
(10) Pub. No.: US 2025/0032051 A1
(43) Pub. Date: Jan. 30, 2025

(54) METHOD AND DEVICE FOR CLASSIFYING ELECTROCARDIOGRAM WAVEFORM BY USING MACHINE LEARNING

(71) Applicant: SEERSTECHNOLOGY CO.,LTD., Seongnam-si, Gyeonggi-do (KR)

(72) Inventors: Youngshin LEE, Yongin-si, Gyeonggi-do (KR); Hoohyun KIM, Guri-si, Gyeonggi-do (KR); Heeseok SONG, Hwaseong-si, Gyeonggi-do (KR); Kunwook CHO, Seongnam-si, Gyeonggi-do (KR)

(73) Assignee: SEERSTECHNOLOGY CO.,LTD., Seongnam-si, Gyeonggi-do (KR)

(21) Appl. No.: 18/715,716

(22) PCT Filed: Dec. 17, 2021
(86) PCT No.: PCT/KR2021/019288

§ 371 Date: Jul. 3, 2024

(30) Foreign Application Priority Data
Dec. 2, 2021 (KR) 10-2021-0170499

(51) Int. Cl. A61B 5/00; A61B 5/352; A61B 5/353; A61B 5/355; A61B 5/363; A61B 5/364

(52) U.S. Cl. CPC A61B 5/7267; A61B 5/352; A61B 5/353; A61B 5/355; A61B 5/363; A61B 5/364; A61B 5/7203; A61B 5/7282

(57) ABSTRACT

Disclosed is an apparatus for electrocardiogram (ECG) wave classification using machine learning being capable of applying a segmentation technique to an ECG wave, checking a feature for each section of a P wave, a Q wave, an R wave, an S wave, a T wave, and a noise wave included in the ECG wave, quickly classifying heart beats into a normal beat (N), a supraventricular beat(S), a ventricular beat (V), and noise, and removing the noise to make medical decisions quickly and accurately.

### TECHNICAL FIELD

The present invention relates to an apparatus for detecting a baseline, a P wave, a QRS (N), a QRS(S), a QRS (V), an R peak, a T wave, and noise in an electrocardiogram and classifying heartbeats using a deep learning-based segmentation algorithm.

### BACKGROUND ART

The information disclosed in this background section is only for enhancement of understanding of the background of the invention, and does not define the prior art.

An electrocardiogram (ECG) reading system that allows a medical staff to analyze an ECG has been developed. A conventional ECG reading system detects R, P, and T peaks of an ECG wave, and detects and classifies arrhythmia in a rule-based manner.

Such an ECG reading system receives patient's entire ECG signal data, analyzes the data, and outputs its result. A deep learning technology has recently been widely studied for an ECG reading algorithm due to its high accuracy.

Only a qualified medical staff can determine arrhythmia using an ECG, but there is a shortage of manpower compared with demand. In reading the ECG, it takes a lot of time since an ECG signal must be read from various perspectives, such as calculation of time differences between shapes and sections of P, QRS, and T waves, and analysis of ECG rhythms. A medical staff must observe a patient's ECG in real time to keep an eye on the patient's condition, but continuous monitoring is difficult due to a lack of manpower. Since the ECG analysis is directly related to the patient's life, the analysis must be performed accurately and quickly in an emergency.

The conventional ECG analysis may use end and start points of P, QRS, and T wave sections, but merely searches for peaks, which results in low usability. In detection and classification of arrhythmia, a typical rule-based algorithm design is less accurate due to diversity of waves, and thus, it is necessary to design a new rule-based algorithm in consideration of arrhythmia.

Particularly, in a case where real-time reading is necessary, such as bedside patient monitoring, it is difficult to use such a conventional ECG reading system. Deep learning algorithms for ECG analysis have different speeds depending on implementation models, and may not easily perform real-time operation every moment. In visualizing the ECG wave, one-dimensional data is output, which lowers readability during real-time reading.

Generally, the ECG wave includes noise generated by a non-invasive bio-signal collection method. The noise included in the ECG waves is a factor that increases a reading time for a medical staff to read the ECG, which results in significant economic losses. In using general automatic reading, in a case where noise is not accurately detected in real-time monitoring, it is difficult to provide accurate reading results to a user. Therefore, there is a need for a technique for increasing the accuracy and speed of reading by accurately detecting and removing noise.

### DISCLOSURE

### Technical Problem

Therefore, the present invention has been made in view of the above problems, and it is an object of the present invention to provide an apparatus for ECG wave classification using machine learning, capable of applying a segmentation technique to an ECG wave, checking a feature for each section of a P wave, a Q wave, an R wave, an S wave, a T wave, and a noise wave included in the ECG wave, quickly classifying heart beats into a normal beat (N), a supraventricular beat(S), a ventricular beat (V), and noise, and removing the noise to make medical decisions quickly and accurately.

### Technical Solution

In accordance with the present invention, the above object can be accomplished by the provision of an ECG noise discriminating apparatus including an ECG wave acquisition unit that acquires an ECG wave for each of a plurality of persons, a classification unit that applies segmentation to the ECG wave to check a feature for each section of the ECG wave, and labels classification values for each section on the basis of the feature, a noise selection unit that selects only a noise wave labeled as noise on the basis of the classification values for each section, and a noise removing unit that removes only the noise wave labeled as the noise.

### Advantageous Effects

As described above, according to this embodiment, it is possible to apply a segmentation technique to an ECG wave to check features of respective sections of a P wave, a Q wave, an R wave, an S wave, a T wave, and a noise wave included in the ECG wave. Further, it is possible to quickly classify heartbeats into a normal beat (N), a supraventricular beat(S), a ventricular beat (V), and noise on the basis of the features, and remove the noise, thereby allowing fast and accurate medical judgment.

According to this embodiment, by learning waves classified as noise using deep learning, it is possible to build a real-time system capable of quickly detecting a noise wave from various ECG waves, and quickly detecting a patient's abnormal state at the moment when medical judgement is needed by quickly removing the noise wave.

According to this embodiment, in a case where a false detection occurs, it is possible to update detection performance of the noise wave by cutting only the relevant section and inputting the section as learning data.

### DESCRIPTION OF DRAWINGS

FIG. 1 is a diagram showing application of semantic segmentation to bio-signal data processing in a bio-signal processing field according to an embodiment of the present invention.

FIG. 2 is a diagram showing point-by-point output of semantic segmentation according to an embodiment of the present invention.

FIG. 3 is a diagram showing ECG semantic segmentation according to an embodiment of the present invention.

FIG. 4 is a diagram showing P, Q, R, S, and T waves (P wave, QRS complex, and T wave) and ECG features according to an embodiment of the present invention.

FIG. 5 is a diagram schematically showing an ECG noise discriminating apparatus according to an embodiment of the present invention.

FIG. 6 is a diagram showing 2D image segmentation and 1D bio-signal segmentation according to an embodiment of the present invention.

FIG. 7 is a diagram showing annotation settings for respective classes according to an embodiment of the present invention.

FIG. 8 is a diagram showing point-by-point annotation according to an embodiment of the present invention.

FIG. 9 is a diagram showing a structure of an encoder-decoder segmentation algorithm according to an embodiment of the present invention.

FIG. 10 is a diagram showing a process of outputting a final output value for each point according to an embodiment of the present invention.

FIG. 11 is a diagram showing a start point and an end point of a T wave according to an embodiment of the present invention.

### DETAILED DESCRIPTION OF THE INVENTION

Hereinafter, the present embodiments will be described in detail with reference to the accompanying drawings.

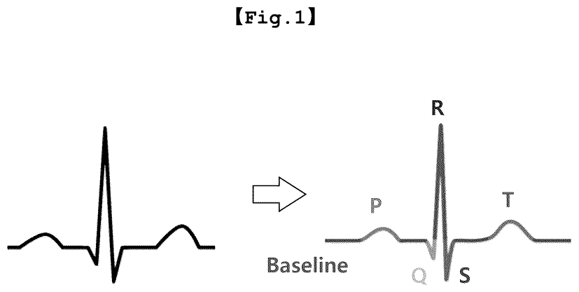

FIG. 1 is a diagram showing application of semantic segmentation to bio-signal data processing in a bio-signal processing field according to an embodiment of the present invention.

An electrocardiogram (ECG) noise discriminating apparatus 500 according to the present embodiment may be applied to one-dimensional (1D) bio-signal data processing in the field of bio-signal processing. The ECG noise discriminating apparatus 500 classifies an ECG wave into a P wave, a QRS-complex (N, S, V), a T wave, and noise included in the ECG wave in the unit of waves, using semantic segmentation.

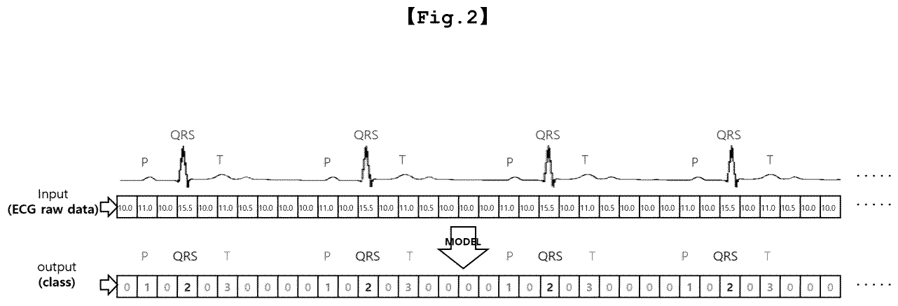

FIG. 2 is a diagram showing point-by-point output of semantic segmentation according to an embodiment of the present invention.

The ECG noise discriminating apparatus 500 according to the present embodiment outputs values obtained by classifying an ECG wave (raw data) in a point-by-point manner using a 1D semantic segmentation algorithm.

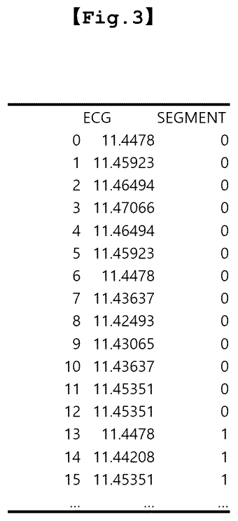

FIG. 3 is a diagram showing ECG semantic segmentation according to an embodiment of the present invention.

In classifying the input ECG wave (raw data) in the point-by-point manner using the 1D semantic segmentation algorithm, the ECG noise discriminating apparatus 500 sets the value as 1 in a case where the wave is noise, and sets the value as 0 in a case where the wave is a normal wave other than noise.

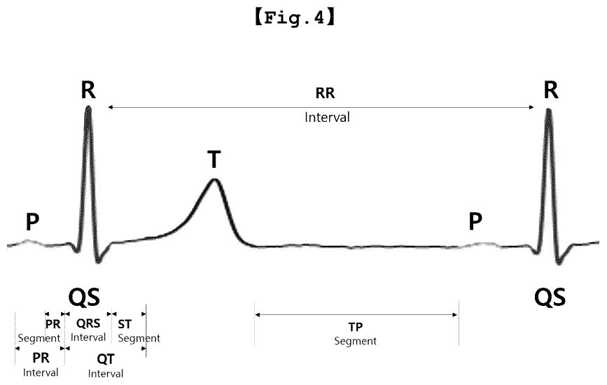

FIG. 4 is a diagram showing P, Q, R, S, and T waves (P wave, QRS complex, and T wave) and ECG features according to an embodiment of the present invention.

The ECG wave appears as a series of beats. The beats may be broadly classified into a normal beat (N), a supraventricular beat(S), and a ventricular beat (V). One beat of the ECG wave basically includes the P wave, the QRS wave, and the T wave.

Further, the ECG wave includes noise generated by a non-invasive bio-signal collection method. The ECG noise discriminating apparatus 500 detects the P wave, the Q wave, the R wave, the S wave, and the T wave included in the ECG wave, and classifies the beats into the normal beat (N), the supraventricular beat(S), and the ventricular beat (V). The ECG noise discriminating apparatus 500 calculates features from localization of the P wave, the Q wave, the R wave, the S wave, and the T wave.

The ECG noise discriminating apparatus 500 distinguishes a PR interval, a QRS interval, a QT interval, an ST segment, and an RR interval in the input ECG wave.

The ECG noise discriminating apparatus 500 may classify the beats on the basis of feature information of the P wave, the Q wave, the R wave, the S wave, and the T wave, on the basis of the PR interval, the QRS interval, the QT interval, the ST segment, and the RR interval.

As described above, the ECG noise discriminating apparatus 500 classifies heart beats into the normal beat (N), the supraventricular beat(S), and the ventricular beat (V). In reading the ECG wave, the ECG noise discriminating apparatus 500 may detect an abnormal state on the basis of the localization and classification information.

The ECG noise discriminating apparatus 500 performs localization on the ECG wave and calculates features.

Then, the ECG noise discriminating apparatus 500 designs several types of arrhythmia detection machine learning models using the features (for example, A arrhythmia detection using features A, B, and D, B arrhythmia detection using features B, C, E, and F, and abnormal beat detection using features A and F).

The ECG noise discriminating apparatus 500 may perform precise localization using segmentation, and obtain reliable and precise features s on the basis of the localization results. The ECG noise discriminating apparatus 500 may detect other abnormal beats, arrhythmias, and the like on the basis of the features.

As described above, the ECG noise discriminating apparatus 500 performs classification on the ECG wave, and classifies heart beats into the normal beat (N), the supraventricular beat(S), and the ventricular beat (V), to thereby detect arrhythmia.

The ECG noise discriminating apparatus 500 applies a segmentation technique to the ECG wave to check features of respective sections of the P wave, the Q wave, the R wave, the S wave, the T wave, and the noise wave included in the ECG wave. The ECG noise discriminating apparatus 500 quickly classifies heart beats into the normal beat (N), the supraventricular beat(S), the ventricular beat (V), and noise on the basis of the features, and removes the noise, thereby allowing fast and accurate medical judgment.

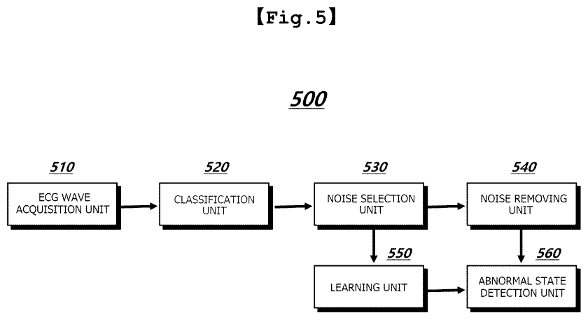

FIG. 5 is a diagram schematically showing an ECG noise discriminating apparatus according to an embodiment of the present invention.

The ECG noise discriminating apparatus 500 according to the present embodiment includes an ECG wave acquisition unit 510, a classification unit 520, a noise selection unit 530, a noise removing unit 540, a learning unit 550, and an abnormal state detection unit 560. The components included in the ECG noise discriminating apparatus 500 are not necessarily limited thereto.

The respective components included in the ECG noise discriminating apparatus 500 may be connected to a communication path that connects software modules or hardware modules within the apparatus to be operated in combination. These components perform communication using one or more communication buses or signal lines.

Each t of the ECG noise discriminating apparatus 500 shown in FIG. 5 refers to a unit that processes at least one function or operation, and may be implemented as a software module, a hardware module, or a combination of software and hardware.

The ECG wave acquisition unit 510 acquires ECG waves for multiple persons. The ECG wave acquisition unit 510 acquires 256 samples of ECG waves per second for each of the multiple persons. The ECG wave acquisition unit 510 converts the ECG waves of the multiple persons into 1D data.

The classification unit 520 applies segmentation to each ECG wave, and confirms a feature of each section of the ECG wave. The classification unit 520 labels a classification value for each section on the basis of the feature.

The classification unit 520 checks feature of the respective sections of the P wave, the Q wave, the R wave, the S wave, the T wave, and the noise wave included in the ECG wave.

The classification unit 520 calculates a noise probability value on the basis of the feature for each section of the ECG wave. The classification unit 520 sets the maximum noise probability value to 1. In a case where the noise probability value is equal to or higher than a threshold, the classification unit 520 determines that the wave is noise, and gives a value of 1, and in a case where the noise probability value is lower than the threshold, the classification unit 520 determines that the wave is a normal wave, and gives a value of 0. The classification unit 520 labels the section with the value of 1 as noise.

In a case where a specific section of the ECG wave is classified as a baseline on the basis of the feature, the classification unit 520 labels the specific section as 0. In a case where a specific section of the ECG wave is classified as the P wave on the basis of the feature, the classification unit 520 labels the specific section as 1. In a case where a specific section of the ECG wave is classified as the normal beat (N) of the QRS complex on the basis of the feature, the classification unit 520 labels the specific section as 2. In a case where a specific section of the ECG wave is classified as the supraventricular beat(S) of the QRS complex on the basis of the feature, the classification unit 520 labels the specific section as 3. In a case where a specific section of the ECG wave is classified as the ventricular beat (V) of the QRS complex on the basis of the feature, the classification unit 520 labels the specific section as 4. In a case where a specific section of the ECG wave is classified as the peak of the R wave on the basis of the feature, the classification unit 520 labels the specific section as 5. In a case where a specific section of the ECG wave is classified as the T wave on the basis of the feature, the classification unit 520 labels the specific section as 6. In a case where a specific section of the ECG wave is classified as the noise wave on the basis of the feature, the classification unit 520 labels the specific section as 7.

The classification unit 520 records values for the P wave, the QRS wave (the normal beat (N), the supraventricular beat(S), and the ventricular beat (V)), and the T wave in the ECG wave as multiple beats.

The noise selection unit 530 selects only the noise wave labeled as noise on the basis of the classification value for each section. The noise removing unit 540 removes only the noise wave labeled as noise.

The learning unit 550 generates an ECG learning result obtained by performing learning using only the noise wave as input. The abnormal state detection unit 560 checks the classification values for the respective sections in time series in a state where the values labeled as noise are removed, and detects an abnormal state (for example, arrhythmia, abnormal beat (S, V), ST, QTc, etc. in a case where the P wave appears again instead of the QRS wave after the P wave).

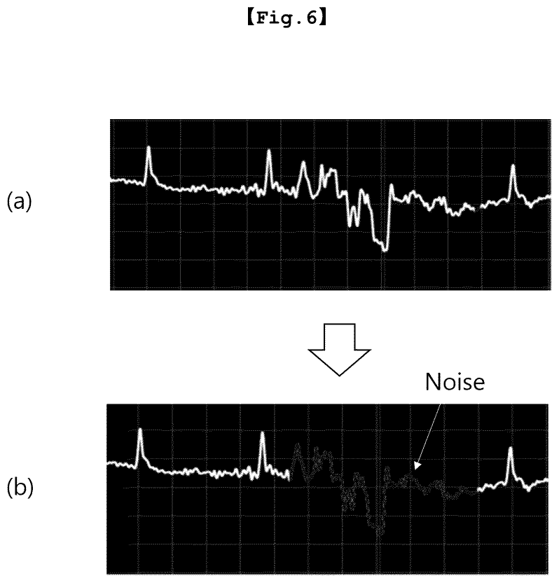

FIG. 6 is a diagram showing 2D image segmentation and 1D bio-signal segmentation according to an embodiment of the present invention.

The ECG noise discriminating apparatus 500 applies the segmentation algorithm to the ECG wave (1D data) to segment the ECG wave into semantic units.

The ECG noise discriminating apparatus 500 detects and classifies the ECG wave using the segmentation algorithm. The ECG noise discriminating apparatus 500 accurately distinguishes sections in the ECG wave using the segmentation algorithm.

As shown in (b) in FIG. 6, in a case where there is a lot of noise mixed in the ECG wave depending on a movement or contact state, the ECG noise discriminating apparatus 500 may remove the noise by applying the segmentation algorithm in one dimension.

The ECG noise discriminating apparatus 500 may learn various types of noise classified using the segmentation algorithm, and may remove the various types of noise generated in the ECG wave on the basis of the learned result.

The ECG noise discriminating apparatus 500 acquires 256 samples per second in order to determine noise. The ECG noise discriminating apparatus 500 determines whether a sample is noise or not as 1 or 0. In order to determine whether the sample is noise, the ECG noise discriminating apparatus 500 sets the maximum noise probability value as 1, and determines a high probability value as noise and a low probability value as normal.

The ECG noise discriminating apparatus 500 may detect noise not only in the ECG but also in various bio-signals using the segmentation algorithm.

(a) in FIG. 6 shows raw signal data. (b) in FIG. 6 shows a state where a certain section is classified into a specific class by a model (the section is annotated as the corresponding class).

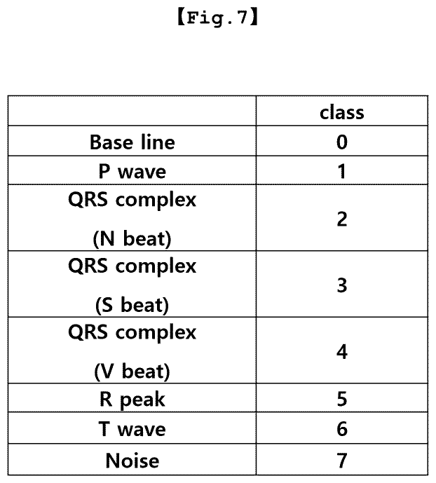

FIG. 7 is a diagram showing annotation settings for respective classes according to an embodiment of the present invention.

The ECG noise discriminating apparatus 500 applies the segmentation technique to the ECG wave to check and classify features of the respective sections of the P wave, the Q wave, the R wave, the S wave, the T wave, and the noise wave included in the ECG wave.

In a case where a classification result obtained by applying the segmentation technique to the ECG wave represents a baseline, the ECG noise discriminating apparatus 500 labels the specific section as 0.

In a case where a specific section of the ECG wave is classified as the P wave on the basis of the feature, using the segmentation technique, the ECG noise discriminating apparatus 500 labels the specific section as 1. In a case where a specific section of the ECG wave is classified as the normal beat (N) of the QRS complex on the basis of the feature, using the segmentation technique, the ECG noise discriminating apparatus 500 labels the specific section as 2. In a case where a specific section of the ECG wave is classified as the supraventricular beat(S) of the QRS complex on the basis of the feature, using the segmentation technique, the ECG noise discriminating apparatus 500 labels the specific section as 3. In a case where a specific section of the ECG wave is classified as the ventricular beat (V) of the QRS complex on the basis of the feature, using the segmentation technique, the ECG noise discriminating apparatus 500 labels the specific section as 4. In a case where a specific section of the ECG wave is classified as the peak of the R wave on the basis of the feature, using the segmentation technique, the ECG noise discriminating apparatus 500 labels the specific section as 5. In a case where a specific section of the ECG wave is classified as the T wave on the basis of the feature, using the segmentation technique, the ECG noise discriminating apparatus 500 labels the specific section as 6. In a case where a specific section of the ECG wave is classified as the noise wave on the basis of the feature, using the segmentation technique, the ECG noise discriminating apparatus 500 labels the specific section as 7.

The ECG noise discriminating apparatus 500 classifies important waves in the ECG wave using the segmentation algorithm, and records the results as multiple labels. The ECG noise discriminating apparatus 500 records values for the P wave, the QRS wave (N, S, V), and the T wave in the ECG wave as multiple beats. For example, the ECG noise discriminating apparatus 500 assigns a total of 7 labels.

The ECG noise discriminating apparatus 500 performs the classification into the P wave, the QRS wave (N, S, V), the T wave, and the noise wave, using the segmentation algorithm. The ECG noise discriminating apparatus 500 performs learning using the segmentation algorithm to distinguish the P wave, the QRS wave (N, S, V), the T wave, and the noise wave in the ECG wave, and labels input data for the learning for each time index.

In performing the learning, the ECG noise discriminating apparatus 500 performs deep learning using input waves, and creates a learning model on the basis of the result. The ECG noise discriminating apparatus 500 may check the labeled numbers to post-facto check whether there is a problem in the ECG (for example, a case where the P wave appears again instead of the QRS wave after the P wave) only with the order of the numbers. The ECG noise discriminating apparatus 500 may check the ECG problem by checking indicators (labeled numbers) without finding peaks.

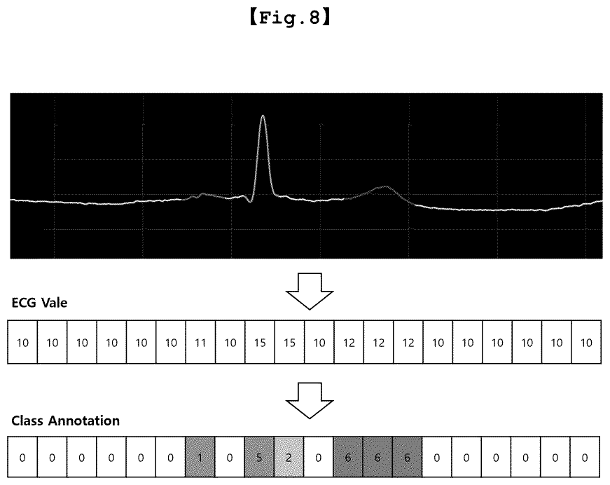

FIG. 8 is a diagram showing point-by-point annotation according to an embodiment of the present invention.

The ECG noise discriminating apparatus 500 prepares only a wave classified as a noise wave as input data for a deep learning model. The ECG noise discriminating apparatus 500 generates learning results using raw data and correct answer data corresponding to the raw data as input data for the deep learning model. The ECG noise discriminating apparatus 500 performs annotation for the correct answer data in respective classes in a point-by-point manner.

The ECG noise discriminating apparatus 500 may reflect one-dimensional ECG values on a graph image and output the result. The ECG noise discriminating apparatus 500 continuously and accumulatively updates the annotated data in the respective classes in the point-by-point manner.

In the process of applying the segmentation algorithm to the ECG wave, the noise discriminating apparatus 500 uses ECG wave data obtained by converting two-dimensional data into one dimensional data as input data for the segmentation algorithm.

Since the ECG noise discriminating apparatus 500 uses noise learning results obtained by learning noise data, it is possible to accurately classify data as noise without concern of classification as normal data.

The ECG noise discriminating apparatus 500 separately labels the noise data, and converts the result into input data that is usable for learning. The ECG noise discriminating apparatus 500 classifies, in a case where there is a noise section in the middle of the ECG wave, the section as noise using the segmentation algorithm.

In a case where the ECG noise discriminating apparatus 500 removes the noise from the ECG wave data using the segmentation algorithm, data to be analyzed is reduced, thereby making it possible to reduce a computational time for analyzing the ECG wave.

Contrarily, in a case where noise detection is not performed using the segmentation algorithm, noise is recognized as a beat in the ECG wave, which frequently results in misreading.

In this case, it takes a lot of time to perform a separate secondary reading and manually correct the misreading result. As described above, according to the present embodiment, the ECG noise discriminating apparatus 500 performs noise detection using the initial segmentation algorithm, and thus, it is possible to process noise-labeled data not to be used in subsequent steps.

In other words, the ECG noise discriminating apparatus 500 primarily determines whether input initial data includes noise using an annotation tool. The ECG noise discriminating apparatus 500 performs deep learning on data determined as noise, and detects noise in an ECG wave that is subsequently input using the learned result.

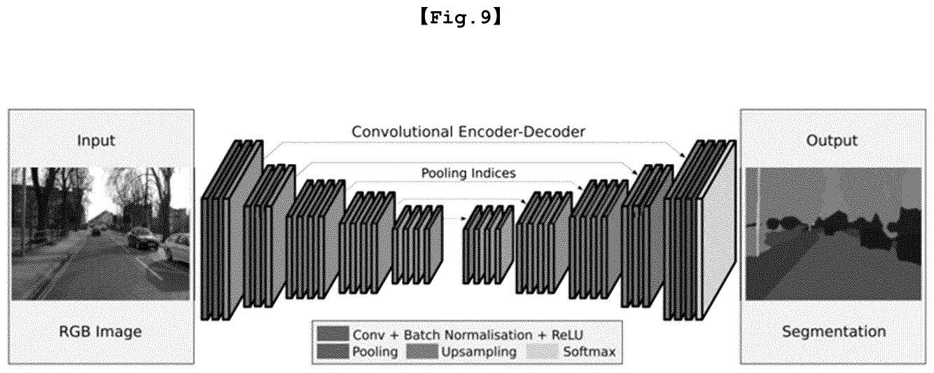

FIG. 9 is a diagram showing a structure of an encoder-decoder segmentation algorithm according to an embodiment of the present invention.

The ECG noise discriminating apparatus 500 generates a segmentation model by performing training using an encoder-decoder type segmentation algorithm. The ECG noise discriminating apparatus 500 detects noise from time-series data by performing semantic segmentation of deep learning. The ECG noise discriminating apparatus 500 detects a noise wave using an AI segmentation algorithm, and removes the noise wave.

Conventionally, in order to detect noise, certain signal processing (for example, FFT (Fast Fourier Transform)) must be performed, but since there are a variety of patterns of noise, it is difficult to actually remove noise due to limitation of a threshold for classification.

The ECG noise discriminating apparatus 500 according to the present embodiment may learn various noise patterns using the segmentation algorithm to detect noise in the ECG wave.

The ECG noise discriminating apparatus 500 uses auto encoder segmentation. In a case where a two-dimensional image is input as input data, the ECG noise discriminating apparatus 500 learning to encode feature vectors.

The ECG noise discriminating apparatus 500 performs decoding so as to perform labeling appropriate for output using a decoder. The ECG noise discriminating apparatus 500 performs compression using an encoder, and performs decoding using the decoder to perform labeling appropriate for output.

After learning raw data using a previously trained learning model, the ECG noise discriminating apparatus 500 determines noise, and labels its corresponding section as noise.

The ECG noise discriminating apparatus 500 performs segmentation in the unit of 1/256 second, and sets a segmentation result value as 1 in a case where the segmentation result is noise, and sets the segmentation result value as 0 in a case where the segmentation result is not the noise. The ECG noise discriminating apparatus 500 reads a section marked as 1 among the segmentation result values as a noise section, and may perform reading excluding the noise section when analyzing the ECG wave later.

The ECG noise discriminating apparatus 500 classifies beats in the ECG wave, and performs reading excluding the noise section (section marked as 1) in the process of reading rhythms and movements for two or more beats.

In performing analysis on a valid wave in the ECG wave, the ECG noise discriminating apparatus 500 performs reading excluding the noise section (section marked as 1). In analyzing the ECG wave, the ECG noise discriminating apparatus 500 checks one-dimensional values labeled according to the time-series indexes, and performs reading excluding the noise section (section marked as 1).

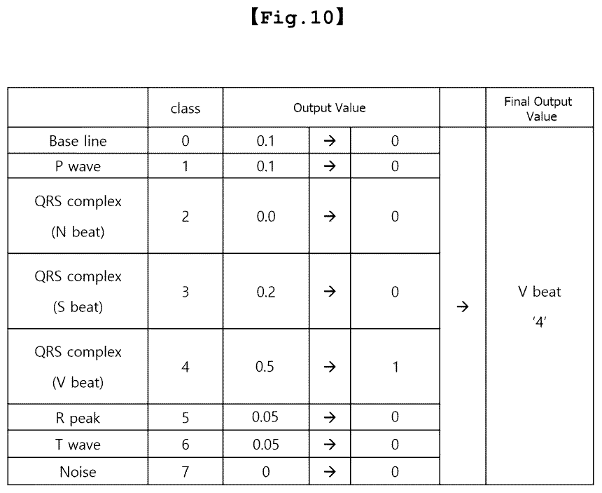

FIG. 10 is a diagram showing a process of outputting a final output value for each point according to an embodiment of the present invention.

The ECG noise discriminating apparatus 500 outputs classification probability values for each class for each section of the ECG wave using the segmentation model. The ECG noise discriminating apparatus 500 determines, as a final output, a class having the highest classification probability value in each class for each section of the ECG wave.

The ECG noise discriminating apparatus 500 simultaneously outputs noise together with a baseline, N, S, V and R peaks as the segmentation result.

The ECG noise discriminating apparatus 500 outputs all probability values of preset classes for one point using the segmentation model (the sum of all the probability values is 1), and finally classifies the point V (4) having the highest probability value as the corresponding point. Here, 256 points make up 1 second, and 2560 points make up a section of 10 seconds. As described above, the ECG noise discriminating apparatus 500 determines noise on the basis of a deep learning result rather than a threshold.

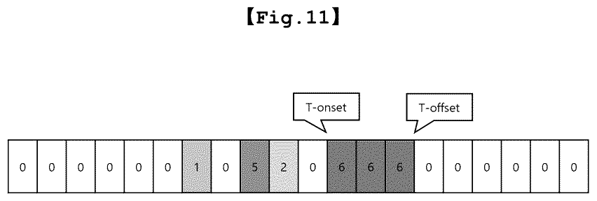

FIG. 11 is a diagram showing a start point and an end point of a T wave according to an embodiment of the present invention.

Since the ECG noise discriminating apparatus 500 outputs a final output value in the point-by-point manner, it is possible to recognize a start point and an end point for each section. The ECG noise discriminating apparatus 500 may recognize the classes for the respective sections at once. The ECG noise discriminating apparatus 500 may analyze an ECG wave in real time using a segmentation algorithm with a lightweight structure.

The ECG noise discriminating apparatus 500 reads noise in the ECG wave using the segmentation algorithm, and records the result data according to a labeled one-dimensional index. The ECG noise discriminating apparatus 500 may record a label as 1 or 0 depending on whether the reading result is noise or not, or may record ECG voltage data.

In a case where the ECG voltage data (raw data) is input to the segmentation algorithm, the ECG noise discriminating apparatus 500 outputs an appropriate label value. In a case where the ECG voltage data (raw data) is input, the ECG noise discriminating apparatus 500 generates a feature vector using the segmentation algorithm, determines whether there is noise on the basis of the feature vector, and labels the result value.

The above description is merely an illustrative explanation of the technical idea of the present embodiment, and those skilled in the art will be able to make various modifications and variations without departing from the scope of the invention. Accordingly, the present embodiments are not intended to limit the technical idea of the invention, and the scope of the technical idea of the invention is not limited by these examples. The scope of protection of the invention should be interpreted in accordance with the claims below, and all 1 equivalent technical ideas should be interpreted as being included in the scope of the invention.

### DESCRIPTION OF REFERENCE NUMERALS

500: ECG NOISE DISCRIMINATING APPARATUS
510: ECG WAVE ACQUISITION UNIT
520: CLASSIFICATION UNIT
530: NOISE SELECTION UNIT
540: NOISE REMOVING UNIT
550: LEARNING UNIT
560: ABNORMAL STATE DETECTION UNIT

1. An electrocardiogram (ECG) noise discriminating apparatus comprising:
an ECG wave acquisition unit that acquires an ECG wave for each of a plurality of persons;
a classification unit that applies segmentation to the ECG wave to check a feature for each section of the ECG wave, and labels classification values for each section on the basis of the feature;
a noise selection unit that selects only a noise wave labeled as noise on the basis of the classification values for each section; and
a noise removing unit that removes only the noise wave labeled as the noise.

2. The apparatus according to claim 1, wherein the classification unit checks features of respective sections of a P wave, a Q wave, an R wave, an S wave, a T wave, and the noise wave included in the ECG wave.

3. The apparatus according to claim 1, wherein the classification unit calculates a noise probability value on the basis of the feature for each section of the ECG wave, sets the noise probability value to a maximum value of 1, discriminates, in a case where the noise probability value is equal to or higher than a threshold, that the section corresponds to the noise and gives a value of 1, and discriminates, in a case where the probability value is lower than the threshold, that the section is not the noise and gives a value of 0.

4. The apparatus according to claim 3, wherein the classification unit labels the section having the value of 1 as the noise.

5. The apparatus according to claim 1, further comprising: a learning unit that generates an ECG learning result by performing learning using only the noise wave as input.

6. The apparatus according to claim 1, wherein the classification unit labels, in a case where a specific section of the ECG wave is classified as a baseline on the basis of the feature, the specific section as 0, labels, in a case where a specific section of the ECG wave is classified as the P wave on the basis of the feature, the specific section as 1, labels, in a case where a specific section of the ECG wave is classified as the peak of the R wave on the basis of the feature, the specific section as 5, labels, in a case where a specific section of the ECG wave is classified as the T wave on the basis of the feature, the specific section as 6, and labels, in a case where a specific section of the ECG wave is classified as the noise wave on the basis of the feature, the specific section as 7.

7. The apparatus according to claim 1, wherein the classification unit labels, in a case where a specific section of the ECG wave is classified as a normal beat (N) of a QRS complex on the basis of the feature, the specific section as 2, labels, in a case where a specific section of the ECG wave is classified as a supraventricular beat(S) of the QRS complex on the basis of the feature, the specific section as 3, and labels, in a case where a specific section of the ECG wave is classified as a ventricular beat (V) of the QRS complex on the basis of the feature, the specific section as 4.

8. The apparatus according to claim 1, wherein the ECG wave acquisition unit acquires 256 samples of the ECG waves per second for each of the plurality of persons.

9. The apparatus according to claim 1, wherein the ECG wave acquisition unit converts the ECG waves of the plurality of persons into one-dimensional data.

10. The apparatus according to claim 1, wherein the classification unit records values for the P wave, the QRS wave (the normal beat (N), the supraventricular beat(S), and the ventricular beat (V)), and the T wave in the ECG wave as multiple beats.

11. The apparatus according to claim 1, further comprising: an abnormal state detection unit that checks the classification values for each section in time series in a state where the value labeled as the noise is removed to determine an abnormal state.
